(19) United States
(12) Patent Application Publication
(10) Pub. No.: US 2024/0181099 A1
(43) Pub. Date: Jun. 6, 2024

(54) ULTRAVIOLET LIGHT IRRADIATION SYSTEM AND ULTRAVIOLET LIGHT IRRADIATION METHOD

(71) Applicant: Nippon Telegraph and Telephone Corporation, Tokyo (JP)

(72) Inventors: Tomohiro TANIGUCHI, Musashino-shi, Tokyo (JP); Satoshi Narikawa, Musashino-shi, Tokyo (JP); Ayako IWAKI, Musashino-shi, Tokyo (JP); Takahito KIRIHARA, Musashino-shi, Tokyo (JP); Kazuhide Nakajima, Musashino-shi, Tokyo (JP); Takashi Matsui, Musashino-shi, Tokyo (JP); Nobutomo HANZAWA, Musashino-shi, Tokyo (JP); Yuto SAGAE, Musashino-shi, Tokyo (JP); Chisato Fukai, Musashino-shi, Tokyo (JP)

(21) Appl. No.: 18/279,424

(22) PCT Filed: Mar. 3, 2021
(86) PCT No.: PCT/JP2021/008270

§ 371 Date: Aug. 30, 2023

(51) Int. Cl. A61L 2/10; A61L 2/26; G02B 6/42

(52) U.S. Cl. CPC A61L 2/10; A61L 2/26; G02B 6/4204; G02B 6/4249; A61L 2202/11

(57) ABSTRACT

An object of the present invention is to provide an ultraviolet light irradiation system and an ultraviolet light irradiation method, each of which is capable of reducing deterioration over time of transmission loss characteristics of an optical fiber due to ultraviolet rays and preventing a decrease in ultraviolet ray irradiation power.

An ultraviolet light irradiation system 301 according to the present invention includes: an ultraviolet light source unit 11 configured to generate ultraviolet rays; and N irradiation units 13 each configured to irradiate a desired location Ar with ultraviolet rays, N being a natural number, wherein a propagation section 50 and a supply section 51 are provided between the ultraviolet light source unit 11 and the irradiation units 13, the ultraviolet ray generated by the ultraviolet light source unit 11 is propagated by space-division multiplexing in the propagation section 50, and the ultraviolet rays subjected to space-division multiplexing in the propagation section 50 are multiplexed and propagated to the irradiation units 13 through a single-core optical fiber in the supply section 51.

### TECHNICAL FIELD

The present disclosure relates to an ultraviolet (UV) light irradiation system and decontamination each of which perform sterilization and virus inactivation using UV rays.

### BACKGROUND ART

For the purpose of preventing infectious diseases, there is an increasing demand for a system that performs sterilization and virus inactivation using UV rays that using UV rays. In the present embodiment, the term “decontamination” includes sterilization and virus inactivation.

There are roughly three categories of products for decontamination systems.

### (1) Mobile Sterilizing Robot

Mobile sterilizing robots are autonomous mobile robots emitting UV rays. The mobile sterilizing robot can automatically achieve decontamination in a wide range without human intervention by emitting UV rights while moving in a room in a building, such as a hospital room. Refer to, for example, the Kantum Ushikata website (https://www.kantum.co.jp/product/sakkin_robot/sakkinn_robot/UVD_robot).

### (2) Stationary Air Purifier

Stationary air purifiers are devices installed on a ceiling or at a predetermined place in a room to circulate and decontaminate air in the room. The stationery air purifier does not emit UV rays to the outside and affect the human body, and thus highly reliable contamination is expected. Refer to, for example, the IWASAKI Electric website (https://www.iwasaki.co.jp/optics/sterilization/air/air03.html).

### (3) Portable Sterilization Device

Portable sterilization devices are portable devices equipped with UV light sources such as florescent lamps, mercury lamps or LEDs. A user takes the portable sterilization device to an area where decontamination is desired, and irradiates the area with UV rays. The portable sterilization device can be used in various places. Refer to, for example, the Funakoshi website (https://www.funakoshi.co.jp/contents/68182).

### CITATION LIST

### Non Patent Literature

Non Patent Literature 1: T. Baba et al., “Development of Heat Resistant UV Transmission Optical Fibers (2)”, Mitsubishi Cable Industries Journal, No. 100, pp. 84-88, April 2003

### SUMMARY OF INVENTION

### Technical Problem

However, the prior art has difficulties as follows:
(1) Since the mobile sterilizing robot emits high-output UV rays, a large-scale and expensive structure is required. Accordingly, the mobile sterilizing robot has economic barriers to implementation.
(2) Since the stationary air purifier adopts sterilization of the circulated indoor air, it is difficult to decontaminate clothes and to immediately remove bacteria and viruses emitted from carriers.
(3) The portable sterilization device has challenges that it emits relatively weak UV rays and thus it is difficult to perform decontamination in a short time. Even if a high-output mercury lamp or fluorescent lamp is employed, these lamps are generally large and have a shorter service life, and further, light is diffused in proportion to the square of the distance, leading to reduced power. Therefore, they are not suitable for the portable sterilization device.

As a solution for the challenges (1) to (3) stated above, optical fiber-based systems have been proposed. By transmitting UV rays from a light source using a thin and bendable optical fiber, it is possible to have flexibility to irradiate a desired place with UV rays output from a fiber tip with enhanced accuracy. Further, a P-MP system configuration used in FTTH (fiber-to-the-home) can lead to higher economic performance by sharing a single light source.

However, the optical fiber-based system has a problem of deterioration in transmission characteristics of the fiber due to transmission of UV rays (see, for example, Non Patent Literature 1). By transmitting high energy light in the ultraviolet region, defects occur in a core glass, and the transmission loss characteristics deteriorate over time. Accordingly, the UV ray power emitted from the optical fiber output end is reduced, leading to insufficient decontamination. Although an operation method of replacing a deteriorated optical fiber can also be adopted, there is a possibility that frequent replacement will occur depending on usage conditions, potentially leading to complicated operation, and thus a demand for efficient solutions remains unchanged.

In short, a decontamination system using the conventional optical fiber has challenges that, in a case where UV rays have a high power, the UV ray power would be reduced due to deterioration of transmission loss characteristic over time.

The present invention is intended to solve such challenges, and an object thereof is to provide an ultraviolet light irradiation system and an ultraviolet light irradiation method, each of which is capable of reducing deterioration over time of transmission loss characteristics of an optical fiber due to UV rays and preventing a decrease in UV ray irradiation power.

### Solution to Problem

For achieving the object, the ultraviolet light irradiation system according to the present invention causes transmission through some sections, through which UV rays propagate in space-division multiplexing (SDM).

In particular, an ultraviolet light irradiation system according to the present invention includes:
an ultraviolet light source unit configured to generate ultraviolet rays; and
N irradiation units each configured to irradiate a desired location with ultraviolet rays, N being a natural number,
wherein a propagation section and a supply section are provided between the ultraviolet light source unit and the irradiation units,
the ultraviolet ray generated by the ultraviolet light source unit is propagated by space-division multiplexing in the propagation section, and
the ultraviolet rays subjected to space-division multiplexing in the propagation section are multiplexed and propagated to the irradiation units through a single-core optical fiber in the supply section.

Furthermore, an ultraviolet light irradiation method according to the present invention is an ultraviolet light irradiation method for irradiating a desired location with ultraviolet rays generated from an ultraviolet light source unit by N irradiation units, N being a natural number,
wherein a propagation section and a supply section are provided between the ultraviolet light source unit and the irradiation units,
the ultraviolet ray generated by the ultraviolet light source unit is propagated by space-division multiplexing in the propagation section, and
the ultraviolet rays subjected to space-division multiplexing in the propagation section are multiplexed and propagated to the irradiation units through a single-core optical fiber in the supply section.

The ultraviolet light irradiation system can irradiate a contaminated site with UV rays having sufficient decontamination power by multiplexing UV rays output from the plurality of fibers while mitigating damage to a transmission path by transmitting UV rays having a high energy density from the ultraviolet light source dispersed by the plurality of fibers. Consequently, with the present invention, it is possible to provide an ultraviolet light irradiation system and an ultraviolet light irradiation method, each of which is capable of reducing deterioration over time of transmission loss characteristics of an optical fiber due to ultraviolet rays and preventing a decrease in ultraviolet ray irradiation power.

In the ultraviolet light irradiation system according to the present invention, the ultraviolet light source unit may be configured to include a plurality of light sources, and the ultraviolet ray output from each of the light sources may be subjected to space-division multiplexing in the propagation section.

In the ultraviolet light irradiation system according to the present invention, the ultraviolet light source unit may be configured to include a light branching unit configured to branch the ultraviolet ray, and each branched portion of the ultraviolet ray may be subjected to space-division multiplexing in the propagation section.

The ultraviolet light irradiation system according to the present invention may further include a photosynthesis unit configured to multiplex all the ultraviolet light, which has been subjected to space-division multiplexing and propagated through the propagation section, provided between the propagation section and the supply section in a case where N is 1.

The ultraviolet light irradiation system according to the present invention may further include a photosynthesis distribution unit configured to divide the ultraviolet rays subjected to space-division multiplexing and propagated through the propagation section into N groups, and to multiplex the ultraviolet rays for each group to input the rays to the N single-core optical fibers, provided between the propagation section and the supply section in a case where N is >2.

In the ultraviolet light irradiation system according to the present invention, the photosynthesis distribution unit may be connected in multiple stages.

In the ultraviolet light irradiation system according to the present invention, the propagation section may be an optical cable that bundles any of a solid core optical fiber, a hole assisted optical fiber, a hole structure optical fiber, a hollow core optical fiber, a coupling core type optical fiber, a solid core type multi-core optical fiber, a hole assisted type multi-core optical fiber, a hole structure type multi-core optical fiber, a hollow core type multi-core optical fiber, and a coupling core type multi-core optical fiber, or alternatively, any one of a solid core type multi-core optical fiber, a hole assisted type multi-core optical fiber, a hole structure type multi-core optical fiber, a hollow core type multi-core optical fiber, and a coupling core type multi-core optical fiber.

The respective inventions above may be combined to any possible extent.

### Advantageous Effects of Invention

With the present invention, it is possible to provide an ultraviolet light irradiation system and an ultraviolet light irradiation method, each of which is capable of reducing deterioration over time of transmission loss characteristics of an optical fiber due to ultraviolet rays and preventing a decrease in ultraviolet ray irradiation power.

### BRIEF DESCRIPTION OF DRAWINGS

FIG. 1 is a diagram illustrating an ultraviolet light irradiation system according to the present invention.

FIG. 2 is a diagram illustrating a propagation section of the ultraviolet light irradiation system according to the present invention.

FIG. 3 is a diagram illustrating a cross-section of an optical fiber.

FIG. 4 is a diagram illustrating an ultraviolet light source unit of the ultraviolet light irradiation system according to the present invention.

FIG. 5 is a diagram illustrating a photosynthesis unit of the ultraviolet light irradiation system according to the present invention.

FIG. 6 is a diagram illustrating an ultraviolet light irradiation system according to the present invention.

FIG. 7 is a diagram illustrating a photosynthesis distribution unit of the ultraviolet light irradiation system according to the present invention.

FIG. 8 is a diagram illustrating the ultraviolet light irradiation system according to the present invention.

FIG. 9 is a diagram illustrating an ultraviolet light irradiation method according to the present invention.

### DESCRIPTION OF EMBODIMENTS

An embodiment of the present invention will be described with reference to the accompanying drawings. The embodiment described below is an example of the present invention, and the present invention is not limited to the following embodiment. Components having the same reference numerals in the present specification and the drawings indicate the same components.

### Gist of Invention

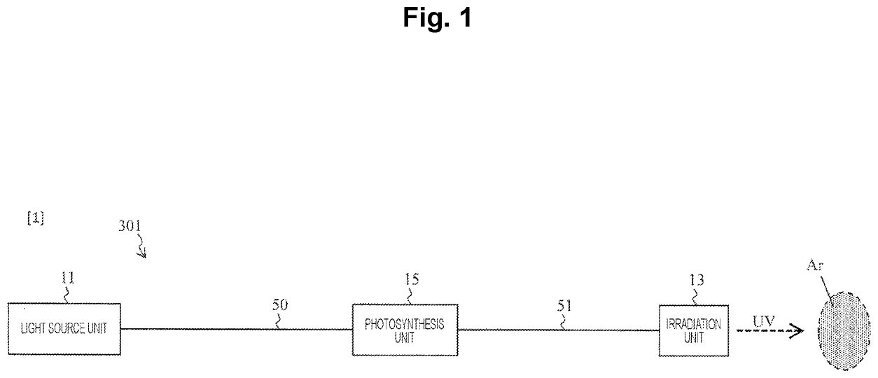

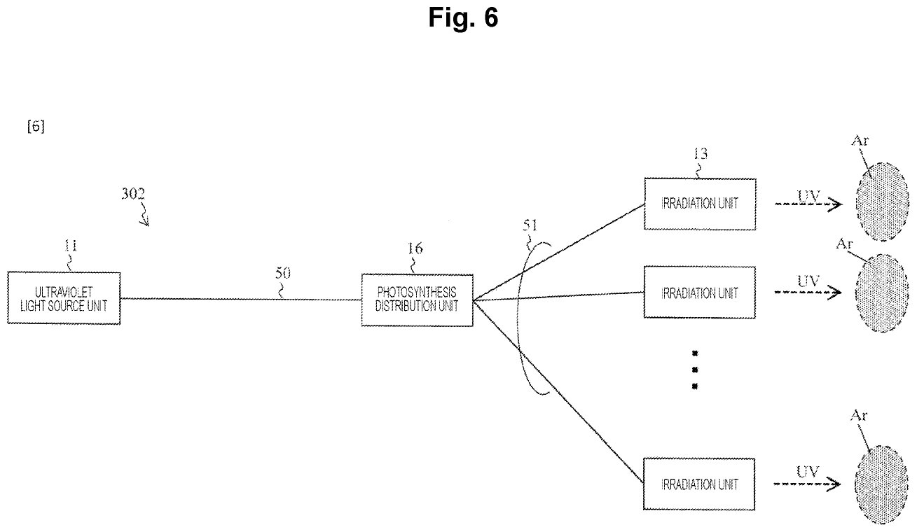

FIGS. 1 and 6 are diagrams illustrating an ultraviolet light irradiation system of the present embodiment.

The ultraviolet light source unit 11 and the irradiation unit 13 installed near a target place Ar to be decontaminated are connected via the photosynthesis unit 15 or the photosynthesis distribution unit 16. In the propagation section 50 in which the optical power of the UV ray is large from the ultraviolet light source unit 11 to the photosynthesis unit 15/photosynthesis distribution unit 16, a plurality of (single core or multi-core) optical fibers are bundled and connected by an optical cable or a multi-core optical fiber. The optical power of the UV ray decreases due to the transmission loss in the propagation section 50. Therefore, the supply section 51 from the photosynthesis unit 15/photosynthesis distribution unit 16 to the irradiation unit 13 can be connected by a single-core optical fiber.

The photosynthesis unit 15 synthesizes output light beams from the plurality of optical fibers or cores and inputs the synthesized light beams to the single-core optical fiber (see a first embodiment described below).

The photosynthesis distribution unit 16 synthesizes output light beams from the plurality of optical fibers or cores, distributes the synthesized light beams, and inputs the light beams to the plurality of single-core optical fibers (see a second embodiment described below).

With this configuration, in the propagation section 50 in which the optical power of the UV ray is large, the entire power is distributed and transmitted to the plurality of optical fibers or cores, so that the problem of characteristic deterioration of each optical fiber or core can be alleviated and efficient operation can be expected.

Furthermore, in the supply section 51, by using a single-core optical fiber having a simple configuration and being thinner, it is possible to implement a system having excellent economic efficiency and capable of being laid in a narrow space.

### First Embodiment

FIG. 1 is a diagram illustrating the ultraviolet light irradiation system 301 of the present embodiment. The ultraviolet light irradiation system 301 includes:
the ultraviolet light source unit 11 configured to generate ultraviolet rays; and
N irradiation units 13 each configured to irradiate a desired location Ar with ultraviolet rays, N being a natural number,
wherein the propagation section 50 and the supply section 51 are provided between the ultraviolet light source unit 11 and the irradiation units 13,
the ultraviolet ray generated by the ultraviolet light source unit 11 is propagated by space-division multiplexing in the propagation section 50, and
the ultraviolet rays subjected to space-division multiplexing in the propagation section 50 are multiplexed and propagated to the irradiation units 13 through a single-core optical fiber in the supply section 51.

The ultraviolet light irradiation system 301 further includes the photosynthesis unit 15 configured to multiplex all the ultraviolet light, which has been subjected to space-division multiplexing and propagated through the propagation section 50, provided between the propagation section 50 and the supply section 51 in a case where N is 1.

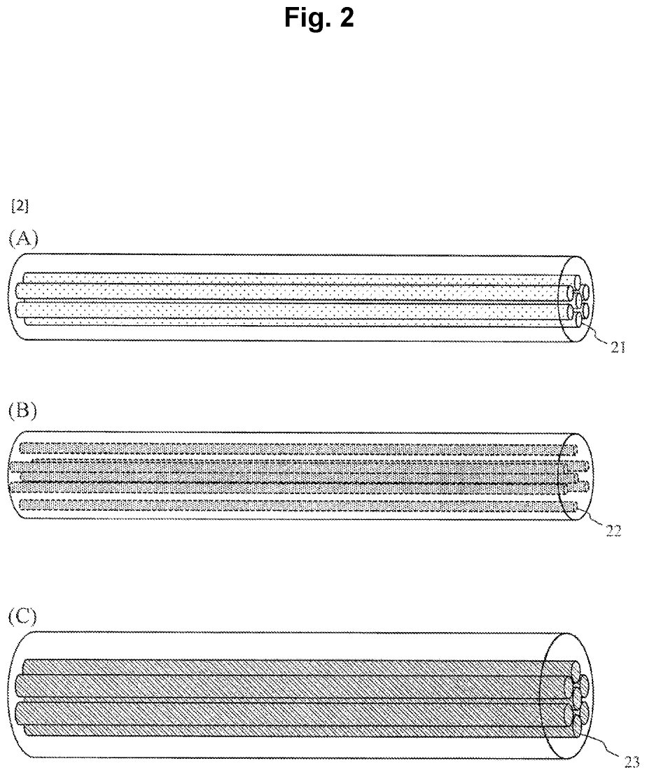

FIG. 2 is a diagram illustrating an optical cable or a multi-core optical fiber constituting the propagation section 50. FIG. 2(A) illustrates an optical cable in which a plurality of single-core optical fibers 21 are bundled. FIG. 2(B) illustrates a multicore optical fiber having a plurality of cores 22. FIG. 2(C) illustrates an optical cable in which a plurality of multi-core optical fibers 23 are bundled.

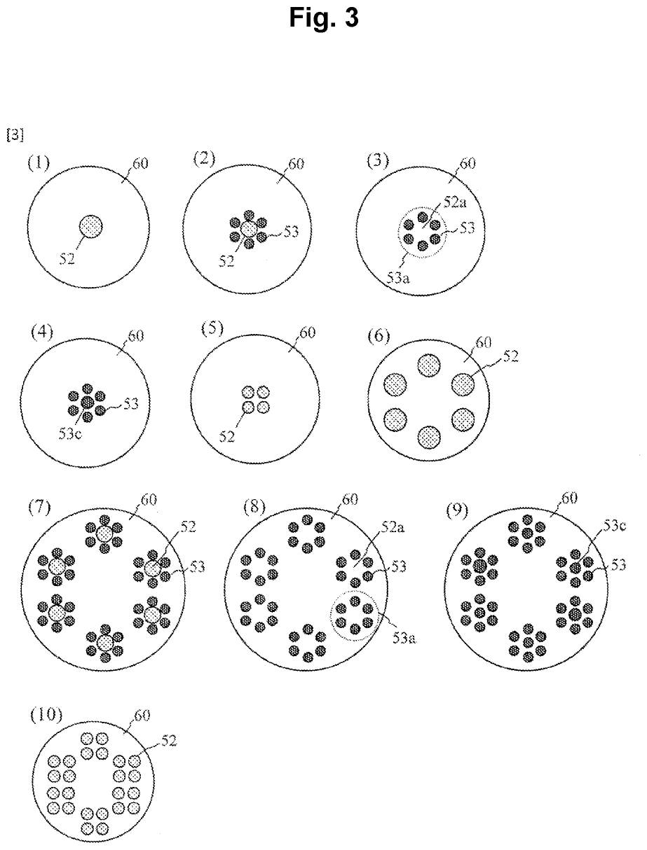

FIG. 3 is a diagram illustrating cross sections of the single-core optical fiber and the multi-core optical fiber described above.

That is, the optical cable of the single-core optical fiber or the multi-core optical fiber, or the multi-core optical fiber, illustrated in FIG. 3, can be used as the propagation section 50. In addition to the solid optical fiber using a general additive as illustrated in FIG. 3(1), the solid optical fiber may be an optical fiber having a hole structure illustrated in FIGS. 3(2) to 3(4), a multi-core optical fiber having a plurality of core regions illustrated in FIGS. 3(5) and 3(6), or an optical fiber having a structure obtained by combining these optical fibers (FIGS. 3(7) to 3(10)).

### (1) Solid Core Optical Fiber

This optical fiber has one solid core 52 having a refractive index higher than that of a clad 60 in the clad 60. “Solid” means “not hollow”. Furthermore, the solid core can also be realized by forming an annular low refractive index region in the clad.

### (2) Hole Assisted Optical Fiber

The optical fiber has the solid core 52 and a plurality of holes 53 arranged on the outer periphery thereof, in the clad 60. The medium of the hole 53 is air, and the refractive index of the air is sufficiently smaller than that of quartz-based glass. Therefore, the hole assisted optical fiber has a function of returning light leaked from the core 52 by bending or the like to the core 52 again, and has a small bending loss.

### (3) Hole Structure Optical Fiber

This optical fiber has a hole group 53a of the plurality of holes 53 in the clad 60, and has a refractive index effectively lower than that of a host material (glass or the like). This structure is called a photonic crystal fiber. This structure can have a structure in which a high refractive index core having a changed refractive index does not exist, and light can be confined using a region 52a surrounded by the holes 53 as an effective core region. Compared with an optical fiber having a solid core, the photonic crystal fiber can reduce the influence of absorption and scattering loss due to additives in the core, and can realize optical characteristics that cannot be realized by a solid optical fiber, such as reduction of bending loss and control of a non-linear effect.

### (4) Hollow Core Optical Fiber

In this optical fiber, a core region is formed of air. Light can be confined in the core region by adopting a photonic band gap structure configured with a plurality of holes or an anti-resonance structure configured with a thin glass wire in the clad region. This optical fiber has a small nonlinear effect, and can supply a high-power or high-energy laser.

### (5) Coupling Core Type Optical Fiber

In this optical fiber, a plurality of solid cores 52 having a high refractive index are arranged close to each other in the clad 60. This optical fiber guides light between the solid cores 52 by optical wave coupling. Since the coupling core type optical fiber can disperse and transmit light by the number of cores, the power can be increased accordingly and efficient sterilization can be performed. In addition, the coupling core type optical fiber has an advantage that fiber degradation due to ultraviolet rays can be alleviated and the life can be extended.

### (6) Solid Core Type Multi-core Optical Fiber

In this optical fiber, the plurality of solid cores 52 having a high refractive index are arranged apart from each other in the clad 60. This optical fiber guides light in a state where the influence of optical wave coupling can be ignored by sufficiently reducing the optical wave coupling between the solid cores 52. Therefore, the solid core type multi-core optical fiber has an advantage that each core can be treated as an independent waveguide.

### (7) Hole Assisted Type Multi-core Optical Fiber

This optical fiber has a structure in which a plurality of hole structures and the core regions of (2) described above are arranged in the clad 60.

### (8) Hole Structure Type Multi-core Optical Fiber

This optical fiber has a structure in which a plurality of hole structures of (3) described above are arranged in the clad 60.

### (9) Hollow Core Type Multi-core Optical Fiber

This optical fiber has a structure in which a plurality of hole structures of (4) described above are arranged in the clad 60.

### (10) Coupling Core Type Multi-core Optical Fiber

This optical fiber has a structure in which a plurality of coupling core structures of (5) described above are arranged in the clad 60.

A propagation mode in these optical fibers may be not only a single mode but also a multi-mode.

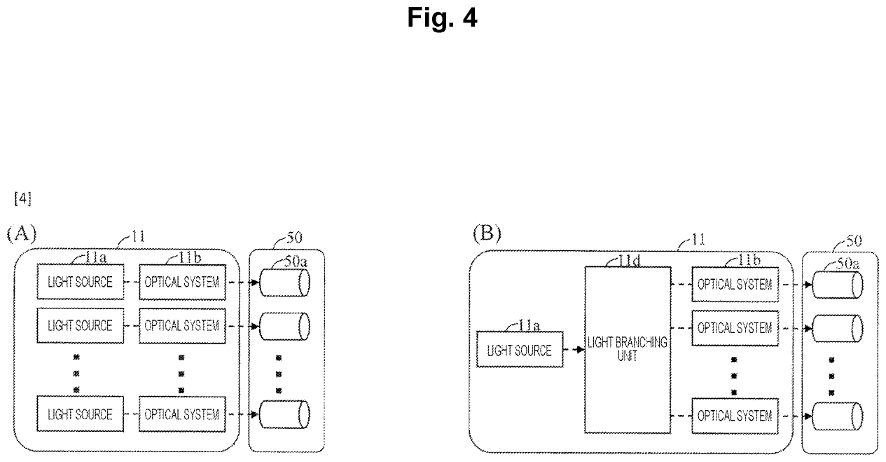

FIG. 4(A) is a diagram illustrating one example of the configuration of the ultraviolet light source unit 11. The ultraviolet light source unit 11 includes a plurality of light sources 11a, and spatially divides and multiplexes the UV ray output from each light source 11a into the propagation section 50.

The light source 11a is a semiconductor light source such as a laser diode (LD) or a light emitting diode (LED), a light source using nonlinear optics as in the following reference, or a lamp light source. [Reference] USHIO website, https://www.ushio.co.jp/jp/technology/lightedge/200012/100236.html

An optical system 11b is, for example, a lens. The optical system 11b inputs the output light of each light source 11a into the optical fiber or core of the propagation section 50.

A propagation path 50a is a single fiber in the optical cable, or a single core in the multi-core optical fiber.

As shown in FIG. 4(A), in a case where the ultraviolet light source unit 11 has a configuration including the plurality of light sources 11a, the output level is not limited to an output level of a single light source, and a system capable of transmitting high power in total can be provided.

FIG. 4(B) is a diagram illustrating another example of the configuration of the ultraviolet light source unit 11. The ultraviolet light source unit 11 includes a light branching unit 11d to branch the ultraviolet ray, and each branched portion of the ultraviolet ray is subjected to space-division multiplexing in the propagation section 50.

In a case of this configuration, the number of light sources 11a is one. The light source 11a, the optical system 11b, and the transmission path 50a are the same as the light source, the optical system, and the transmission path described in FIG. 4(A), respectively.

The light branching unit 11d branches the UV ray output from the light source 11a and enters the plurality of optical systems 11b.

As shown in FIG. 4(B), when the ultraviolet light source unit 11 is configured to branch the output of a single light source 11a, the ultraviolet light source unit 11 can have a simple configuration because there is only one single light source.

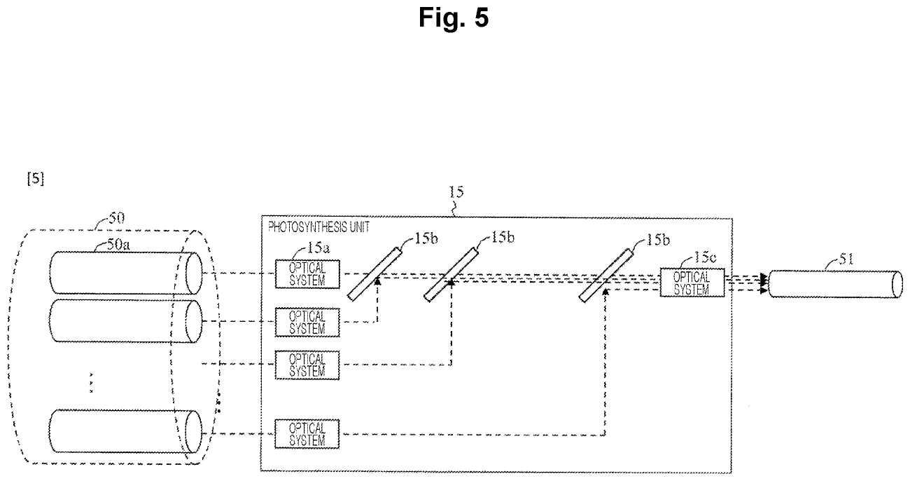

FIG. 5 is a diagram illustrating one example of the configuration of the photosynthesis unit 15. The photosynthesis unit 15 has an optical system 15a for each transmission path 50a, a beam combiner 15b for each transmission path 50a, and one optical system 15c. The optical system 15a is, for example, a lens, and collimates the UV ray from the transmission path 50a to output to the beam combiner 15b. The beam combiner 15b has a function of synthesizing the transmitted light and the reflected light, and multiplexes the UV ray from each optical system 15a and outputs it to the optical system 15c. The optical system 15c is, for example, a lens, and the multiplexed UV ray enters into the single-core optical fiber of the supply section 51. That is, the photosynthesis unit 15 synthesizes the UV rays from the plurality of optical fibers or cores and inputs the synthesized UV rays to the single-core optical fiber.

The supply section 51 transmits UV ray to the irradiation unit 13. The supply section 51 is a single-core optical fiber, has a simple configuration and is excellent in economic efficiency, and is thin and thus can be laid in a narrow space. The optical fiber described in (1) to (5) of FIG. 3 can be used as the single-core optical fiber of the supply section 51.

The irradiation unit 13 irradiates a predetermined target site Ar to be sterilized with the UV ray transmitted through the optical cable or the multi-core optical fiber. The irradiation unit 13 includes an optical system such as a lens designed for a wavelength in the ultraviolet region.

With the configuration stated above, the ultraviolet light irradiation system 301 can reduce deterioration over time of transmission loss characteristics of the optical fiber due to UV rays and prevent a decrease in UV ray irradiation power.

### Second Embodiment

FIG. 6 is a diagram illustrating an ultraviolet light irradiation system 302 of the present embodiment. The ultraviolet light irradiation system 302 includes:
the ultraviolet light source unit 11 configured to generate ultraviolet rays; and
N irradiation units 13 each configured to irradiate a desired location Ar with ultraviolet rays, N being a natural number,
wherein the propagation section 50 and the supply section 51 are provided between the ultraviolet light source unit 11 and the irradiation units 13,
the ultraviolet ray generated by the ultraviolet light source unit 11 is propagated by space-division multiplexing in the propagation section 50, and
the ultraviolet rays subjected to space-division multiplexing in the propagation section 50 are multiplexed and propagated to the irradiation units 13 through a single-core optical fiber in the supply section 51.

The ultraviolet light irradiation system 301 further includes: the photosynthesis distribution unit 16 configured to divide the ultraviolet rays subjected to space-division multiplexing and propagated through the propagation section 50 into N groups, and to multiplex the ultraviolet rays for each group to input the rays to the N single-core optical fibers, provided between the propagation section 50 and the supply section 51 in a case where N>2.

In the present embodiment, only a configuration different from the ultraviolet light irradiation system 301 described in FIG. 1 will be described. The ultraviolet light irradiation system 302 is different from the ultraviolet light irradiation system 301 in including the plurality of irradiation units 13 since there are the plurality of decontamination places Ar and the photosynthesis distribution unit 16 that distributes the UV ray propagated in the propagation section 50 to each irradiation unit 13.

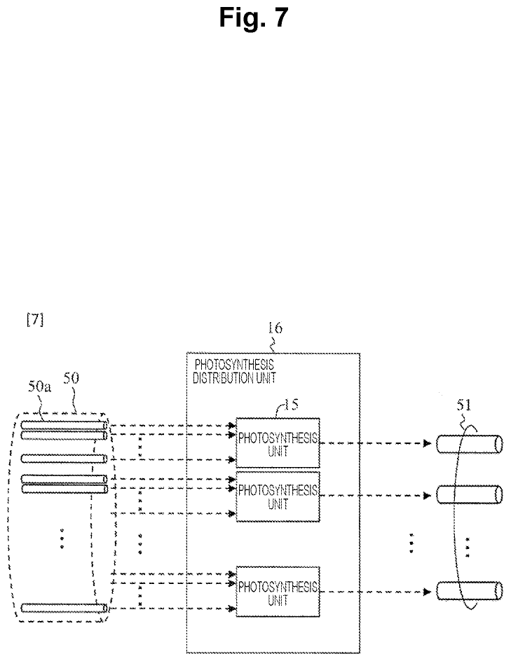

FIG. 7 is a diagram illustrating one example of the configuration of the photosynthesis distribution unit 16. The photosynthesis distribution unit 16 includes N photosynthesis units 15 as described with reference to FIG. 5. Each of the photosynthesis units 15 multiplexes the UV ray from the plurality of transmission paths 50a and outputs the multiplexed light to one single-core optical fiber in the supply section 51. That is, the photosynthesis distribution unit 16 synthesizes output light beams from a plurality of optical fibers or cores for each group, and inputs the synthesized light beams to a single-core optical fiber corresponding to the group.

With the configuration stated above, the ultraviolet light irradiation system 302 can reduce deterioration over time of transmission loss characteristics of the optical fiber due to UV rays and prevent a decrease in UV ray irradiation power. The ultraviolet light irradiation system 302 can irradiate a plurality of decontamination sites with UV rays from the single ultraviolet light source unit, leading to economic advantages.

### Third Embodiment

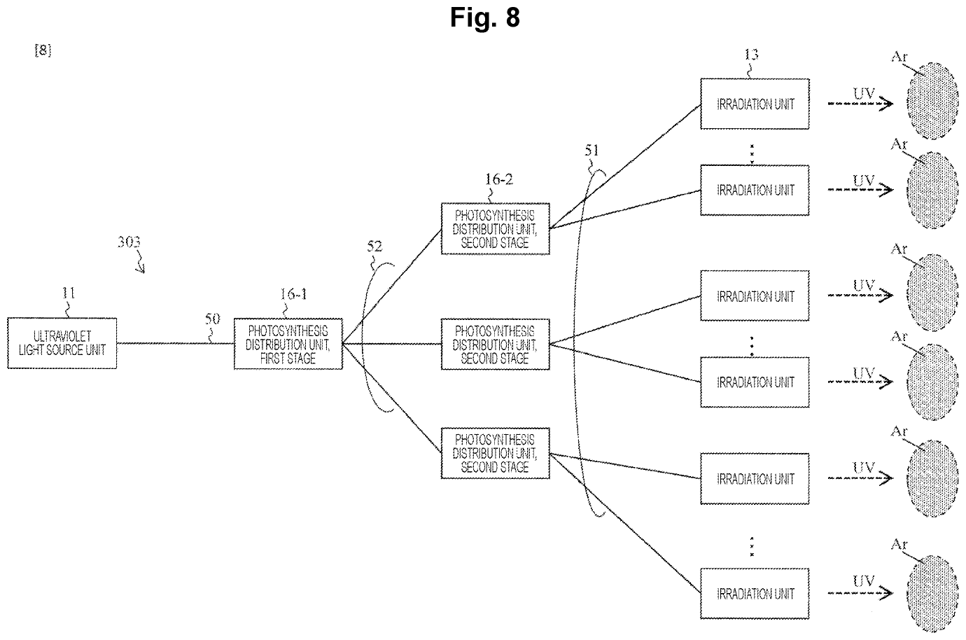

FIG. 8 is a diagram illustrating an ultraviolet light irradiation system 303 of the present embodiment. The ultraviolet light irradiation system 303 is different from the ultraviolet light irradiation system 302 of FIG. 6 in that the photosynthesis distribution unit 16 is connected in multiple stages. FIG. 8 illustrates an example in which the photosynthesis distribution unit 16 has two stages. The photosynthesis distribution unit 16-1 and the photosynthesis distribution unit 16-2 of each stage have the same configuration as that of the photosynthesis distribution unit 16 described in FIG. 7. The photosynthesis distribution unit 16-1 and the photosynthesis distribution unit 16-2 are connected by the optical cable or the multi-core optical fiber (propagation section 52) described in FIG. 2. The photosynthesis distribution unit 16-1 multiplexes the UV ray in the propagation section 50 for each group, and inputs each ray to the optical fiber of the optical cable, or the core of the multi-core optical fiber in the propagation section 52.

With the configuration stated above, the ultraviolet light irradiation system 303 can reduce deterioration over time of transmission loss characteristics of the optical fiber due to UV rays and prevent a decrease in UV ray irradiation power. The ultraviolet light irradiation system 303 can irradiate a plurality of decontamination sites with UV rays from the single ultraviolet light source unit, leading to economic advantages.

### Fourth Embodiment

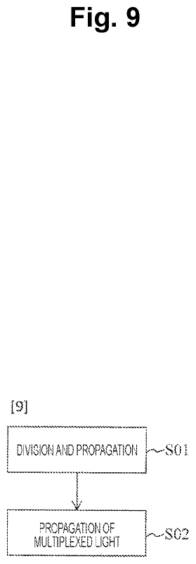

FIG. 9 is a flowchart illustrating an ultraviolet light irradiation method using the ultraviolet light irradiation system (301, 302, or 303) of the present embodiment. An ultraviolet light irradiation method according to the present invention is an ultraviolet light irradiation method for irradiating a desired location Ar with ultraviolet rays generated from an ultraviolet light source unit 11 by N irradiation units 13, N being a natural number,
wherein a propagation section 50 and a supply section 51 are provided between the ultraviolet light source unit 11 and the irradiation units 13,
the ultraviolet ray generated by the ultraviolet light source unit 11 is propagated by space-division multiplexing in the propagation section 50 (step S01), and
the ultraviolet rays subjected to space-division multiplexing in the propagation section are multiplexed and propagated to the irradiation units 13 through a single-core optical fiber in the supply section 51 (step S02).

### REFERENCE SIGNS LIST

11 Ultraviolet light source unit
11a Light source
11b Optical system
11d Light branching unit
13 Irradiation unit
15 Photosynthesis unit
15a Optical system
15b Beam combiner
15c Optical system
50 Propagation section
50a Transmission path
51 Supply section
52 Solid core
52a Region
53 Hole
53a Hole group
60 Cladding
301 to 303 Ultraviolet light irradiation system

1. An ultraviolet light irradiation system, comprising:
an ultraviolet light source unit configured to generate ultraviolet rays; and
N irradiation units each configured to irradiate a desired location with ultraviolet rays, N being a natural number,
wherein a propagation section and a supply section are provided between the ultraviolet light source unit and the irradiation units,
the ultraviolet ray generated by the ultraviolet light source unit is propagated by space-division multiplexing in the propagation section, and
the ultraviolet rays subjected to space-division multiplexing in the propagation section are multiplexed and propagated to the irradiation units through a single-core optical fiber in the supply section.

2. The ultraviolet light irradiation system according to claim 1,
wherein the ultraviolet light source unit is configured to include a plurality of light sources, and
the ultraviolet ray output from each of the light sources is subjected to space-division multiplexing.

3. The ultraviolet light irradiation system according to claim 1,
wherein the ultraviolet light source unit is configured to include a light branching unit configured to branch the ultraviolet ray, and
each branched portion of the ultraviolet ray is subjected to space-division multiplexing.

4. The ultraviolet light irradiation system according to claim 1, further comprising a photosynthesis unit configured to multiplex all the ultraviolet light, which has been subjected to space-division multiplexing and propagated through the propagation section, provided between the propagation section and the supply section in a case where N is 1.

5. The ultraviolet light irradiation system according to claim 1, further comprising a photosynthesis distribution unit configured to divide the ultraviolet rays subjected to space-division multiplexing and propagated through the propagation section into N groups, and to multiplex the ultraviolet rays for each group to input the rays to the N single-core optical fibers, provided between the propagation section and the supply section in a case where N is >2.

6. The ultraviolet light irradiation system according to claim 5, wherein the photosynthesis distribution unit is connected in multiple stages.

7. The ultraviolet light irradiation system according to claim 1, wherein the propagation section is an optical cable that bundles any of a solid core optical fiber, a hole assisted optical fiber, a hole structure optical fiber, a hollow core optical fiber, a coupling core type optical fiber, a solid core type multi-core optical fiber, a hole assisted type multi-core optical fiber, a hole structure type multi-core optical fiber, a hollow core type multi-core optical fiber, and a coupling core type multi-core optical fiber, or alternatively, any one of a solid core type multi-core optical fiber, a hole assisted type multi-core optical fiber, a hole structure type multi-core optical fiber, a hollow core type multi-core optical fiber, and a coupling core type multi-core optical fiber.

8. An ultraviolet light irradiation method for irradiating a desired location with ultraviolet rays generated from an ultraviolet light source unit by N irradiation units, N being a natural number,
wherein a propagation section and a supply section are provided between the ultraviolet light source unit and the irradiation units,
the ultraviolet ray generated by the ultraviolet light source unit is propagated by space-division multiplexing in the propagation section, and
the ultraviolet rays subjected to space-division multiplexing in the propagation section are multiplexed and propagated to the irradiation units through a single-core optical fiber in the supply section.

9. The ultraviolet light irradiation method according to claim 8,
wherein the ultraviolet light source unit is configured to include a plurality of light sources, and
the ultraviolet ray output from each of the light sources is subjected to space-division multiplexing.

10. The ultraviolet light irradiation method according to claim 8,
wherein the ultraviolet light source unit is configured to include a light branching unit configured to branch the ultraviolet ray, and
each branched portion of the ultraviolet ray is subjected to space-division multiplexing.

11. The ultraviolet light irradiation method according to claim 8, further comprising multiplexing all the ultraviolet light, which has been subjected to space-division multiplexing and propagated through the propagation section, provided between the propagation section and the supply section in a case where N is 1.

12. The ultraviolet light irradiation method according to claim 8, further comprising using a photosynthesis distribution unit to divide the ultraviolet rays subjected to space-division multiplexing and propagated through the propagation section into N groups, and to multiplex the ultraviolet rays for each group to input the rays to the N single-core optical fibers, provided between the propagation section and the supply section in a case where N is >2.

13. The ultraviolet light irradiation method according to claim 12, wherein the photosynthesis distribution unit is connected in multiple stages.

14. The ultraviolet light irradiation method according to claim 8, wherein the propagation section is an optical cable that bundles any of a solid core optical fiber, a hole assisted optical fiber, a hole structure optical fiber, a hollow core optical fiber, a coupling core type optical fiber, a solid core type multi-core optical fiber, a hole assisted type multi-core optical fiber, a hole structure type multi-core optical fiber, a hollow core type multi-core optical fiber, and a coupling core type multi-core optical fiber, or alternatively, any one of a solid core type multi-core optical fiber, a hole assisted type multi-core optical fiber, a hole structure type multi-core optical fiber, a hollow core type multi-core optical fiber, and a coupling core type multi-core optical fiber.
